(19) United States
(12) Patent Application Publication
(10) Pub. No.: US 2018/0193248 A1
(43) Pub. Date: Jul. 12, 2018

(54) DETERMINATION OF MELANIN INHIBITION POTENTIAL OF NATURAL INGREDIENTS

(71) Applicant: Kemin Industries, Inc., Des Moines, IA (US)

(72) Inventors: Naresh Modepalli, West Des Moines, IA (US); Satish Nayak, West Des Moines, IA (US)

(21) Appl. No.: 15/401,677

(22) Filed: Jan. 9, 2017

(51) Int. Cl. A61K 8/92; A61Q 19/02

(52) U.S. Cl. CPC A61K 8/922; A61K 2800/782; A61Q 19/02

(57) ABSTRACT

A method of reducing the level of melanin in the skin of a subject is disclosed wherein an efficacious amount of Chinese Lantern oil is applied to the skin.

### RELATED APPLICATIONS

This application claims priority to U.S. Patent Application Ser. No. 62/276,071, filed Jan. 7, 2016, which is incorporated herein in its entirety by this reference.

### BACKGROUND OF THE INVENTION

Melanogenesis is a complex process of melanin pigment synthesis by large dendritic cells known as melanocytes, which are located at the epidermal dermal junction in the skin^{1,2}. Pigmentation of tissues depends on the amount, type, and distribution of melanin. The amount of melanin in our skin determines our phenotypic appearance^{3,4}. Pigmentary disorders are the third most common dermatologic disorders and have a significant psychosocial distress among many ethnic groups⁵. The accumulation of abnormal melanin (i.e. hyperpigmentation) in skin is an aesthetic problem among a large segment of the adult population. Hyperpigmentation is a common problem caused by various inflammatory skin disorders such as eczema, dermatitis and results in conditions such as melasma, solar lentigines and post-inflammatory hyperpigmentation⁶. Routine exposure to daylight (i.e. sun light), intrinsic aging and various other biological and environmental factors also contribute to hyperpigmentation⁷.

Development of skin lightening products without causing melanocyte toxicity is one of the major challenges for cosmetic industry. There is an unaddressed quest for safe and effective depigmenting agents, since the traditional depigmenting agents such as hydroquinone, kojic acid and β-arbutin are either not effective in all skin types or they have significant side effects⁸.

Melanin is produced by melanocytes in the skin. Hyperpigmentation generally results from three major steps: the proliferation of melanocytes, the synthesis and activation of tyrosinase to produce melanin, and the transfer of melanosomes from melanocytes to keratinocytes. Inhibition of the tyrosinase enzyme activity could help to reduce melanin production.

### SUMMARY OF THE INVENTION

The aim of the present study was twofold: first to develop in-house assays to measure melanogenesis and second to estimate the skin lightening potential of selective ingredients using these assays, which might serve as natural alternatives to existing hyperpigmenting agents. In the first assay, tyrosinase enzyme inhibition potential of natural ingredients was tested. Tyosinase plays an important role in the biosynthesis of melanin (FIG. 1). Tyrosinase catalyzes hydroxylation of the amino acid tyrosine into DOPA (3,4-dihydroxyphenyl-alanine) and in a second enzymatic step, catalyzes the oxidation of DOPA to dopaquinone. Subsequently, dopaquinone is converted by a series of complex reactions involving cyclization and oxidative polymerizations which finally result in the formation of melanin⁹. Tyrosinase inhibition activity of the tested materials was determined and compared to that of the known benchmarks.

Commercially available natural ingredients were tested for tyrosinase inhibition potential: a natural extract of rosemary containing carnosic acid (Rosamox™, Kemin Industries, Inc., Des Moines, Iowa); Chinese lantern oil (Kemin Industries, Inc.); and lysolecithin (Lysofix™, Kemin Industries, Inc.). In the second assay, melanocyte-keratinocyte co-culture was developed for testing of these natural ingredients to determine their effect on pigmentation and melanogenesis. In the final part of the study, an in vitro 3D tissue model of the human epidermis (MelanoDerm) was used to assess the potential of Rosamox and Chinese lantern oil on tissue pigmentation and melanin synthesis.

From co-culture assay and MelanoDerm assay, Chinese lantern oil at 0.1% concentration showed a significant reduction in the amount of melanin produced compared to the control. From microscopic visualization studies, it was evident that Chinese lantern oil at 0.1% had a strong potential as an effective melanogenesis inhibitor.

### BRIEF DESCRIPTION OF THE DRAWINGS

FIG. 1 is a scheme of the biosynthesis of melanin and role of tryosinase enzyme in melanin pigment production; the initial reactions involving tyrosinase enzyme are the rate-limiting steps in melanin synthesis (adapted from Parvez at al., Phytother. Res. 21, 805-816, 2007¹⁰).

FIG. 2 is a schematic diagram showing the treatment configuration: Tissue is treated with topically applied ingredient; cultures stands [two stainless steel washers (EPI-WSHR)] are stacked one atop another are used to allow the use of 5.0 mL of maintenance medium beneath the cultures. [Image source: MatTek corp, MA].

FIG. 3 is a graph of the percent inhibition of tyrosinase enzyme activity for Rosamox, Lysofix and Chinese lantern oil treatments and positive controls; kojic acid, β-arbutin and hydroquinone are positive controls; the data points represented are the average of n=6±S.D.

FIG. 4 is a chart of the percent inhibition of melanin for different treatments and positive control (Kojic Acid) in co-culture assay; control represents the absolute amount (100%) of melanin produced in co-culture; the data points represented are the average of n=3±S.D. (Means with ‘*’ are statistically significant from control).

FIG. 5 is a chart of the standard calibration curve of synthetic melanin used for melanin determination in MelanoDerm assay; melanin dissolved in Solvable™ reagent to obtain concentrations ranging from 0-100 μg, shown on x-axis and relative absorbance was shown on y-axis.

FIG. 6 is a chart of the relative melanin content for different treatments and positive controls in MelanoDerm assay; the amount of melanin produced with treatments and positive control were compared with control; the data points represented are the average of n=3±S.D. (Means with ‘*’ are statistically significant from control).

### DESCRIPTION OF THE INVENTION

Plants of the Lamiaceae family are known to produce carnosic acid. In particular, rosemary (Rosmarinus officinalis) produces high amounts of carnosic acid which can be extracted via a straight-forward CO₂ extraction. The water extract may be used to produce an oil soluble product with a high concentration, 5% and higher by weight, of carnosic acid.

Chinese lantern (Physalis alkekengi) is a herbaceous perennial plant native to Asia that produces an orange to red fruit enclosed in a larger, bright orange to red papery basal calyx. Chinese lantern is a popular ornamental plant and has been used in traditional medicine as a diuretic, antiseptic, liver corrective and sedative. Chinese lantern calixes have a high level of antioxidant compounds, including carotenoids.

As used herein, “reducing” means treating, ameliorating, reducing the adverse appearance of, reducing the severity of, or reducing the adverse effects of.

As used herein, “therapeutically effective amount” means the amount of a compound or composition or derivatives thereof of the present invention is an amount that, when administered to a subject, will have the intended therapeutic effect. The full therapeutic effect does not necessarily occur by administration of one dose, and may occur only after administration of a series of doses. Thus, a therapeutically effective amount may be administered in one or more administrations. The precise effective amount needed for a subject will depend upon, for example, the subject's size, health and age, the nature and extent of the condition, and the therapeutics or combination of therapeutics selected for administration, and the mode of administration. The skilled worker can readily determine the effective amount for a given situation by routine experimentation. In one embodiment, the extract of Chinese lantern as described herein is added to a personal care product for application to the skin in a therapeutically effective amount when used as directed.

As used herein, “treatment or treating” means intervention in an attempt to alter the natural course of the individual, animal or cell being treated, and may be performed either for prophylaxis or during the course of clinical pathology. Desirable effects include preventing occurrence or recurrence of disease, alleviation of symptoms, diminishment of any direct or indirect pathological consequences of the disease, lowering the rate of disease progression, amelioration or palliation of the disease state, and remission or improved prognosis. A condition or subject refers to taking steps to obtain beneficial or desired results, including clinical results. Beneficial or desired clinical results include, but are not limited to, reduction, alleviation or amelioration of one or more symptoms associated with skin damage.

Fresh Chinese lantern berries were freeze-dried. The obtained dried berries were then ground to produce a powder. This powder was used for extraction of oil. Extraction was conducted using supercritical carbon dioxide. The extraction was conducted using the following conditions: Temperature, 45° C.; pressure, 300 bar; CO₂ flow rate, 100 g/min; and solvent to feed ratio, 70. The obtained extract was an orange-colored oil. In preferred embodiments of the present invention, the dosage of an extract of Chinese lantern oil ranges from 0.001% by weight of a personal care product to 10% by weight of a personal care product and all values between such limits, including, for example, without limitation or exception, 0.002%, 0.003%, 0.004%, 0.01%, 0.03%, 0.06%, 0.09%, 0.1%, 0.25%, 0.7%, 1%, 2%, 3%, 4%, 4.15%, 6.63%, and 9.87%. Stated another way, in preferred embodiments of the invention, the dosage can take any value “ab.cde wt %” wherein a is selected from the numerals 0 and 1, and b, c, d and e are each individually selected from the numerals 0, 1, 2, 3, 4, 5, 6, 7, 8 and 9 with the exception that a, b, c, d and e cannot all be 0.

### Example 1

### Materials and Methods

Chemicals.

Kojic acid (Sigma-Aldrich, Catalog# K3125, Lot# BCBG6172V, expiration date Mar. 5, 2017), β-arbutin (Sigma-Aldrich, Catalog# A4256, Lot# BCBK4993V, expiration date Jan. 5, 2017), hydroquinone (Sigma-Aldrich, Catalog# H9003, Lot# BCBK0157V, expiration date May 5, 2017), Rosamox (in sunflower oil) (product no #018000-02-WW, Lot #1408101448), Lysofix dry (product no #018043-127-WW, Lot #1412104153).

Production of LumiSalis SE.

Chinese Lantern oil [LumiSalis SE lot #592142P5] is a natural oil extracted from Chinese lantern berries using supercritical CO₂ extraction¹¹. Briefly, Chinese lantern berries were harvested and the berries and the sepals were hand separated and stored at a −20° C. freezer before use. The berries were mashed with a Cuisinart DFP-14BCN 14-Cup food processor for 1-2 minutes. The mashed berries were dried using a freeze dryer (DURA-TOP™ bulk tray dryer from FTS system, INC. Warminster, Pa.) to obtain orange red material. The freeze drying cycle started at −20° C. at day 1, and the dryer temperature increased 10° C. daily until it reached 30° C. The vacuum of 40 torr was applied to the system. The drying cycle time was 6 days. The dry material was further ground with a coffee bean grinder for 1-2 minutes to ensure the seeds were ground into powder. The Chinese lantern berry powder was subjected to supercritical CO₂ extraction with a SFT-150 supercritical extractor (Supercritical Fluid Technologies, Newark, Del.). Ten gram of ground Chinese lantern berry powder was weighed in an extraction bag (supplied by Supercritical Fluid Technologies). The bag was then placed in the extraction vessel which was then sealed. The extraction was performed using the following conditions:
Extraction pressure: 300 bar
Extraction temperature: 45° C.
Solvent to feed ratio: 20
Flow rate of CO₂: 10 SCFH (Standard Cubic Feet per Hour)
Extraction time: 1.4 hours
   Once the desired operating conditions were achieved the outlet to the collection vial was opened to obtain a steady flow of extracted material. Once the extract was collected, the outlet valve was shut and the system was depressurized and allowed to cool to ambient pressure and temperature. When ambient conditions were achieved the extraction vessel was opened to remove the spent material. This extraction process yielded 1.08 g of oil. The wax in the oil was removed by low temperature (4° C.) centrifugation using a refrigerated floor centrifuge (Sorvall® RC-5C Plus) at 4000 g for 25 minutes. The final product (1 g) was orange red oil.

Tyrosinase Inhibition Assay.

Rosamox (0.1%, 0.05% and 0.01%), Lysofix dry (0.1%, 0.05% & 0.01%) and Chinese lantern oil (0.1%, 0.05%, 0.01% & 0.005%) were tested for inhibition of tyrosinase enzyme activity. Kojic acid (2%, 1% & 0.5%), β-arbutin (5%, 2%, 1% & 0.5%) and hydroquinone (5%, 2%, 1% & 0.5%) served as positive controls in this study. Rosamox and Chinese lantern oil samples were prepared in Dimethylsulfoxide (DMSO). Lysofix sample, positive controls, 3,4-dihydroxyphenyl-alanine (L-DOPA) and mushroom tyrosinase were prepared using 0.1 M phosphate buffer (pH-6.6).

Tyrosinase inhibition potential of natural ingredients was measured using a modified method published previously by Uchida et al¹². Briefly, the test samples or positive control (20 μL) were added to the reaction mixture containing 0.12 mL of 0.1 M potassium phosphate buffer (pH 6.6) in 96 well microplates and incubated at room temperature for 2 minutes. After 2 minutes of incubation, 40 μL of 10 mM L-DOPA solution was added to the reaction mixture and incubated for 5 minutes at 37° C. using a benchtop incubator (Fisher scientific, PA). Twenty microliters of mushroom tyrosinase (200 units/mL) was added to the above reaction mixture and incubated at 37° C. in a conventional lab incubator (Fisher Scientific, PA) for 10 minutes. The amount of dopachrome produced in the reaction mixture was measured spectrophotometrically using Spectramax Me5 plate reader at 475 nm. The tyrosinase activity and the % inhibition of tyrosinase in presence of test material were calculated as follows:

Tyrosinase activity=(S(Abs)−B(Abs))/C(Abs)×100

Inhibition (%)=1−{(S(Abs)−B(Abs))}/C(Abs)×100 Equation 1.
Formula for Measuring tyrosinase activity and % tyrosinase inhibition, where S(Abs)—sample absorbance; B(Abs)—Blank absorbance (Without enzyme) and C(Abs)—Control Absorbance [Buffer and DMSO].

Cell Culture Models.

Cell culture studies were performed using Human Epidermal Keratinocytes (HEK) continuous cell lines [HEK001 (ATCC® CRL-2404™) lot #61331463] and Primary epidermal melanocytes (ATCC PCS-200-013, lot #60948598). HEK cells were grown in the HEK growth media consisting of keratinocytes serum free medium (GIBCO-BRL 17005042, lot #1638561) with 5 ng/mL human recombinant epidermal growth factor (EGF) (ATCC, lot #1584416) and 2 mM L-glutamine (ATCC, lot #62195752) (without bovine pituitary extract and serum). HEK growth media was supplemented with penicillin (10000 units) and streptomycin (10 mg/mL) solution (Gibco, lot #1469707). Primary epidermal melanocytes growth media consisting of Dermal cell basal medium (ATCC PCS-200-030) with Melanocytes Growth kit [ATCC PCS-200-042] (rh Insulin 5 μg/mL, Ascorbic acid 50 μg/mL, L-glutamine 6 mM, Epinephrine calcium chloride 1.5 μM peptide growth factor and M8 supplement) and supplemented with penicillin (10 units/mL)-streptomycin (10 μg/mL)-Amphotericin B solution (25 ng/mL). Cells were grown in cell culture flasks (75 cm²) [Fisher Scientific, catalog number 10-126-37] to approximately 80% confluence in 5% CO₂ environment incubator (NuAire, MN) maintained at 37° C. HEK cultures and melanocytes were routinely observed visually at the beginning, after media changes and splitting procedures and at the end of each experiment. All experiments were performed with HEK cells at passage no. 98.

Co-Culture Development.

HEK cells were harvested by treatment with trypsin/EDTA solution and resuspended in HEK media. Viable cells were determined by trypan blue exclusion study and counted using a hemocytometer. HEK cells were seeded at 1.25*10⁴ cells/well in twelve-well plates. Next day, primary melanocytes were collected by treatment with trypsin/EDTA solution for primary cells and added to each well (25000 cells) containing the HEK cells. The initial seeding was at 5:1 for HEK:melanocytes. In normal human skin the ratio of keratinocyte:melanocyte ratio is in slight excess of 30:1. In this assay, 5:1 seeding ratio is used to accelerate the effects of whitening agents on the cell layers, minimizing the length of experiments as the cell proliferation rate was very quick and varies between the two cell lines. Co-cultures of melanocytes and keratinocytes were maintained in HEK and melanocytes growth media used at ratio of 5:1 (final volume 1.2 mL). Used culture media was replaced every 24 hours with fresh media.

Determination of Melanin Inhibition of Natural Ingredients Using Co-Culture Cell Model.

Rosamox (0.05% and 0.01%), Lysofix dry (0.05% & 0.01%) and Chinese lantern oil (0.1%, 0.05%, 0.01% & 0.005%) were tested for inhibition of melanin synthesis in this assay. Kojic acid (2%, 1% & 0.5%) was used as positive control. All the samples were prepared using sterile phosphate buffered saline (PBS) with 5% DMSO solution. The treatments were applied on co-culture from day 3 and continued for 5 days along with fresh media. At the end of treatment period, cells were lysed with 200 μL of 1 N sodium hydroxide (NaOH) solution and homogenized by applying stress through repetitive pipetting. The cell extracts were transferred into 96-well plates in triplicate. Relative melanin content in the cell extracts was determined by absorbance spectroscopy at 405 nm using a SpectraMax Me5 plate reader. The amount of melanin produced in the co-culture was measured with the help of standard calibration curve of melanin prepared using synthetic melanin (Sigma-Aldrich, Catalog# M8631). For standard calibration curve, synthetic melanin was dissolved in the cell culture media along with 1 N NaOH to obtain melanin concentrations ranging from 0-25 μg.

Determination of Melanin Inhibition of Natural Ingredients Using Human 3D Skin Tissue Model.

A well-developed 3D tissue model is robust compared to cell monolayers and allows to measure tissue pigmentation changes more relevant to actual physiological conditions. MatTek's (MatTek Corp. MA) MelanoDerm System consists of normal, human derived epidermal keratinocytes (NHEK) and melanocytes (NHM) which have been cultured to form a multilayered, highly differentiated model of the human epidermis. MEL-A (Melanocytes and keratinocytes derived from Asian donor tissue) were used in the current study. The MelanoDerm skin model exhibits in vivo-like morphological and ultrastructural characteristics.

MelanoDerm Tissue Preparation.

Upon arrival, the MelanoDerm tissues (MatTek, Mel-300A, Lot#17489B, expiration date May 1, 2015) were immediately processed for use. The tissues were removed from the agarose-shipping tray and placed into a 6-well plate containing 1.0 mL of assay medium (MatTek, EPI-100-NMM-113, Lot#040915RKE, expiration date May 7, 2015) warmed to 37±2° C. After incubation, cell inserts were transferred to fresh 6-well plates containing culture stands and 5 mL of media.

The test materials (Chinese lantern oil at 0.05% and 0.1% and Rosamox at 0.05% and 0.1%) were prepared in a mixture of 5% DMSO and 95% PBS. Kojic acid at 1% was used as a positive control while the DMSO/PBS vehicle alone was used as a negative control in this assay. Fifty microliters of each test material was applied to the surface of the tissue (FIG. 2) and incubated at 37±2° C. and 5±1% CO₂. Every 24 hours, the tissues were rinsed with PBS and fresh test material was applied. The media was changed on alternative days. The treatments were carried out for 21 days. At the end of treatment period melanin content was measured using Solvable™ reagent (Tissue and Gel Solubilizer 0.5 M) with the help of solvable melanin assay¹³.

Solvable Melanin Assay.

At the end of treatment period, the cell inserts (containing the MelanoDerm tissues) were submerged in phosphate buffered saline (PBS) for 15 minutes to remove any residual phenol red and test material from the tissue. PBS was decanted and tissues were sealed in a plastic bag and frozen (−20±5° C.) over night. Next day, tissues were thawed by placing them at room temperature. Later, cell culture inserts were filled with 1% sodium bicarbonate (−300 μL/insert) and allowed to sit for 30 minutes, and sodium bicarbonate was discarded. Tissues were removed from the inserts using sterile fine-point forceps. Tissues were dried completely and each tissue was placed in a 1.7 mL centrifuge tube. Five hundred microliters of Solvable™ reagent was added to each tube ensuring that the tissue was completely submerged. Centrifuge tubes containing the tissue samples were incubated at 95° C. overnight along with melanin standards prepared from 0-100 μg using standard melanin (Sigma-Aldrich, Catalog# M8631). Melanin stock solution was prepared at 1 mg/mL and Solvable™ reagent was used for dilutions. Samples and standards were mixed well by using a benchtop vortex and allowed to cool for five minutes and centrifuged at 13,000 rpm for 5 minutes to obtain a pellet of tissue leftover. Three hundred microliters of supernatant from each sample and standard were transferred to 96 microwell plates. The plate was read at 490 nm ensuring there were no bubbles or foam in the wells prior to reading. The amount of melanin produced was measured with the help of melanin standard curve prepared using synthetic melanin (0-100 μg) dissolved in Solvable™ reagent.

For visualization of treatment effect on tissue culture, microscopic observation of MelanoDerm tissue on day 1, 4, 10, 14 and finally on day 21 was carried out using glass bottom microwell dish with the help of benchtop inverted microscope (Nikon Inc., NY) at 20× magnification. Images were captured using Tucsen camera (Xintu Photonics Co., China) attached to the microscope.

Statistical Analysis.

Values were reported as means±standard deviations (SD). Data were analyzed using an unpaired t-test (http://graphpad.com/quickcalcs/). All p values were two-tailed and P<0.05 was considered to be significant.

### Results

Tyrosinase Inhibition Assay.

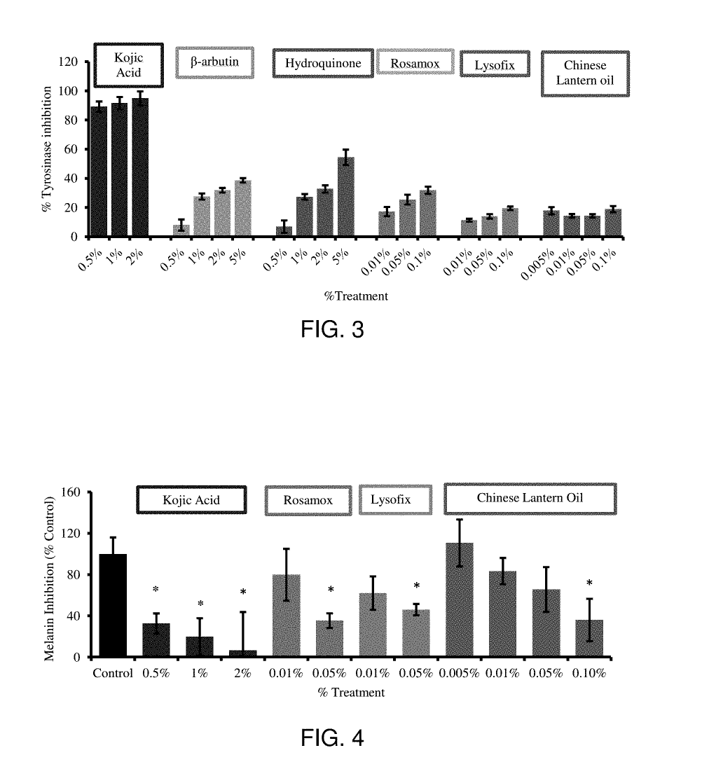

From the tyrosinase inhibition activity of tested samples and positive controls as shown in FIG. 3, among the tested positive controls, kojic acid at all concentrations (0.5%, 1% and 2%) was effective in inhibiting tyrosinase enzyme compared to control. The control was considered to be hundred percent activity of tyrosinase as the reaction mixture contains DOPA and tyrosinase to produce dopachrome at maximum. Hydroquinone and β-arbutin even at high concentrations (5%) were not as effective as kojic acid. Rosamox was tested at 0.01-0.1% concentrations and the % inhibition was ˜20-40% compared to kojic acid at 0.5%. Lysofix dry at tested concentrations inhibited tyrosinase by ˜10-15% and Chinese lantern oil (0.05-0.1%) was effective in inhibiting only 15-20% enzyme compared to kojic acid at 0.5%. Overall, there was only a marginal efficacy of the tested natural materials in inhibiting tyrosinase enzyme at the tested concentrations.

Melanocyte-Keratinocyte Co-Culture Study.

The results from melanocyte-keratinocyte co-culture are shown in FIG. 4. There was a significant reduction in melanin production with kojic acid at tested concentrations with maximum effect at highest tested concentration (2%) compared to control (p<0.005). Rosamox at 0.05% concentration was effective in reducing melanin production significantly compared to the control. Lysofix at 0.05% concentration was effective in reducing melanin production significantly (p<0.005), and at 0.01% there was a minimal reduction in melanin production which was not significant compared to control. Chinese lantern oil at 0.1% concentration was effective in reducing the melanin production significantly (p<0.05) compared to control. But there was no significant difference in melanin production at lower concentrations.

MelanoDerm Assay.

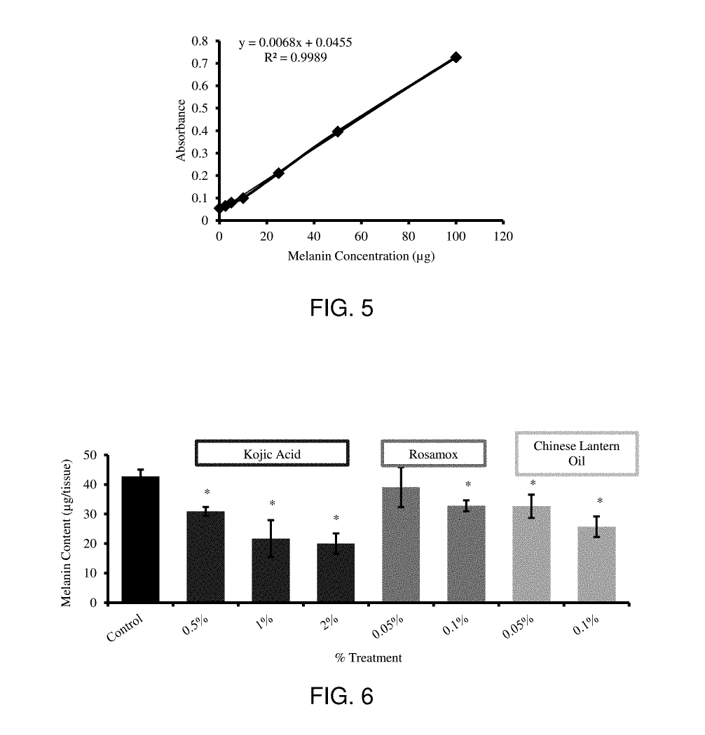

To overcome the limitations of co-culture model, a 3D MelanoDerm model was used to measure melanin synthesis inhibition capabilities of Rosamox and Chinese lantern oil. Melanin standard calibration curve used for melanin determination in this study is shown in FIG. 5. From MelanoDerm assay results (shown in FIG. 6), Rosamox at 0.05% showed minimal inhibition on melanin production compared to control, which was not statistically significant. At 0.1%, the density of melanin granules was less compared to control; this might be due to its mild irritant nature at this concentration. Chinese lantern oil at 0.05% showed some inhibition on melanin production compared to control which was significant (p<0.05). At 0.1% concentration, there was a significant reduction (P<0.005) in the amount of melanin granules produced compared to control and might be acting as an effective melanin synthesis inhibitor. Melanoderm tissues were very sensitive to handle and cost-effective. In this preliminary investigation, we have limited our studies to Rosamox and Chinese lantern using MelanoDerm unlike other assays. The efficacy of Lysofix in Melanoderm tissue will be investigated in future studies.

Microscopic images of MelanoDerm tissues with all samples and standard treatments were taken on day 1, 4, 10, 14 and 21 and reviewed (not shown). From these microscopic images, the melanin content in the control treatment increased gradually showing the presence of dense melanin granules at day 21. Treatment with kojic acid at 0.5%, showing the presence of some melanin granules, but kojic acid at 1% and 2% show no sign of melanin granules at the end of 21 days. Among the treatments, Chinese lantern oil at 0.1% showed fewer melanin granules, supporting the MelanoDerm assay results.

### DISCUSSION

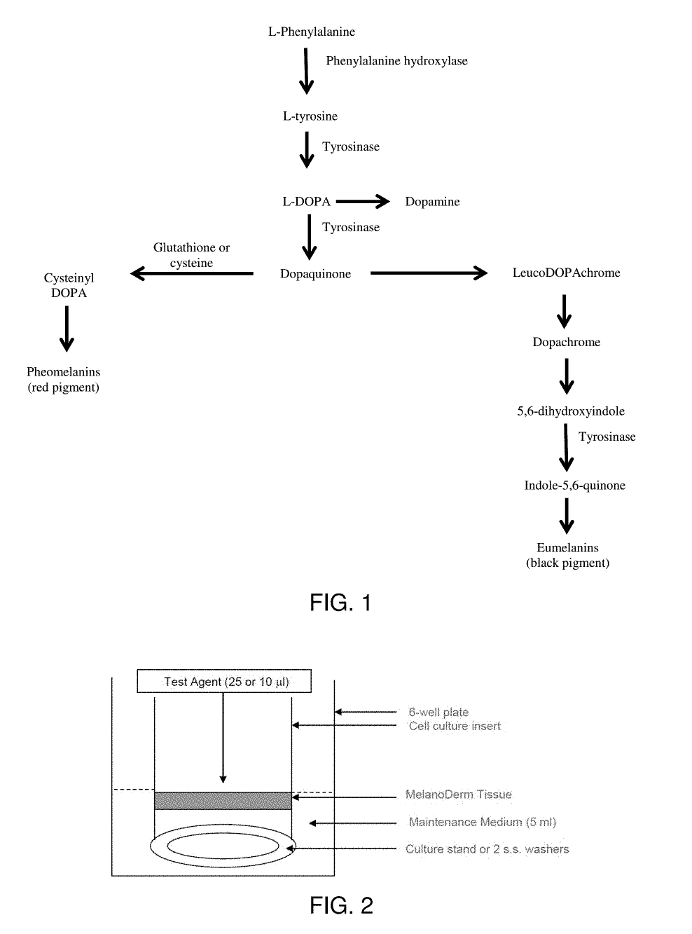

Cell culture based assays were developed for measuring the melanin inhibition potential of natural ingredients. All the tested natural ingredients showed marginal efficacy in tyrosinase enzyme inhibition. As shown in FIG. 1, tyrosinase involves in the first two steps of melanin synthesis and along with tyrosinase activity, various other enzymatically catalyzed chemical reactions are involved in the melanogenesis process. Other than tyrosinase inhibition, skin lightening can be achieved by inhibition of melanosomal transfer and by acceleration of epidermal turnover and desquamation¹⁴. Antioxidants also have been proven to be effective in skin whitening potential.

Chinese lantern oil at 0.1% was proven to be effective in melanin inhibition in both co-culture assay and MelanoDerm assay. Linoleic acid is one of the major active components of Chinese lantern oil¹⁵. The concentration of linoleic acid found in Chinese lantern oil was ˜63%¹⁵. Linoleic acid was reported to be effective in reduction of melanogenesis, and the amount of tyrosinase protein which eventually might help in the reduction of melanogenesis in murine melanoma cell model¹⁶. From earlier studies, Chinese lantern oil exhibited potential benefits by reducing photo-induced inflammation and by down-regulating proteases that degrade extra cellular matrix proteins¹¹ in human dermal fibroblasts and it might also have the capabilities to scavenge free radicals which indirectly helps in skin whitening effects.

Rosamox at 0.05% showed efficacy in inhibiting melanin in co-culture assay, but in MelanoDerm assay this concentration was not effective in inhibiting melanin compared to control. From MelanoDerm results, Rosamox at 0.1% and higher concentrations might have the potential to inhibit melanin but repetitive applications of this active at higher concentrations might lead to irritation issues in individuals with sensitive skin.

The foregoing description and drawings comprise illustrative embodiments of the present inventions. The foregoing embodiments and the methods described herein may vary based on the ability, experience, and preference of those skilled in the art. Merely listing the steps of the method in a certain order does not constitute any limitation on the order of the steps of the method. The foregoing description and drawings merely explain and illustrate the invention, and the invention is not limited thereto, except insofar as the claims are so limited. Those skilled in the art that have the disclosure before them will be able to make modifications and variations therein without departing from the scope of the invention.

### REFERENCES

1. Hearing V J. Biogenesis of pigment granules: a sensitive way to regulate melanocyte function. J Dermatol Sci. 2005; 37:3-14.
2. Curto E V, Kwong C, Hermersdörfer H, Glatt H, Santis C, Virador V, Hearing V J Jr, Dooley T P. Inhibitors of mammalian melanocyte tyrosinase: in vitro comparisons of alkyl esters of gentisic acid with other putative inhibitors. Biochem Pharmacol. 1999; 57(6):663-72.
3. Alaluf S, Barrett K, Blount M et al. (2003) Ethnic variation in tyrosinase and TYRP1 expression in photoexposed and photoprotected human skin. Pigment Cell Res 16:35-42.
4. Solano F, Briganti S, Picardo M and Ghanem G. Hypopigmenting agents: an updated review on biological, chemical and clinical aspects. Pigment Cell Res. 19; 550-571.
5. Halder R M, Nootheti P K. Ethnic skin disorders overview. J Am Acad Dermatol. 2003; 48:S143-8.
6. Leyden J J, Shergill B, Micali G, Downie J, Wallo W. Natural options for the management of hyperpigmentation. J Eur Acad Dermatol Venereol. 2011 October; 25(10): 1140-5.
7. Gilchrest B A, Blog F B and Szabo G. Effects of Aging and Chronic Sun Exposure on Melanocytes in Human Skin. Journal of Investigative Dermatology. 1979; 73, 141-143.
8. Zhu W and Gao J. The Use of Botanical Extracts as Topical Skin-Lightening Agents for the Improvement of Skin Pigmentation Disorders. Journal of Investigative Dermatology Symposium Proceedings. 2008; 13, 20-24.
9. Park H, Song K H, Jung P M, Kim J E, Ro H, Kim M Y and Ma J Y. Inhibitory Effect of Arctigenin from Fructus Arctii Extract on Melanin Synthesis via Repression of Tyrosinase Expression. Evidence-Based Complementary and Alternative Medicine. 2013; id: 965312.
10. Hearing V J Jr, Monophenol monooxygenase (tyrosinase): Purification, properties, and reactions catalyzed. Methods Enzymol 1987; 142: 154-165.
11. Wei S. and Nayak S. The Role of LumiSalis™SE in prevention of photoaging of skin. 2014. WP-15-00003.
12. Uchida R, Seiko Ishikawa S and Tomoda H. Inhibition of tyrosinase activity and melanine pigmentation by 2-hydroxytyrosol. Acta Pharmaceutica Sinica B 2014; 4(2):141-145
13. MEL-300-A-B-C_Protocol MK-24-007-0019.
14. Gillbro J M and Olsson M J. The melanogenesis and mechanisms of skin-lightening agents—existing and new approaches. International Journal of Cosmetic Science, 2011; 33: 210-221.
15. Wei S. Shen M and Nayak S. Bioactives from Chinese lantern berries: extraction, composition and efficacy. 2014. PRE-14-00159.
16. Ando H, Funasaka Y, Oka M, Ohashi A, Furumura M, Matsunaga J, Matsunaga N, Hearing V J, and Ichihashi M. Possible involvement of proteolytic degradation of tyrosinase in the regulatory effect of fatty acids on melanogenesis. J Lipid Res. 1999; 40(7):1312-6.

We claim:

1. A method of reducing the level of melanin in the skin of a subject, comprising the step of administering an efficacious amount of Chinese lantern oil.

2. The method of claim 1, further comprising a personal care product and wherein said amount of Chinese lantern oil ranges from 0.001% by weight of the personal care product to 10% by weight of the personal care product.

3. A method of reducing melanogenesis in the skin of a subject, comprising the step of administering an efficacious amount of Chinese lantern oil.

4. The method of claim 3, further comprising a personal care product and wherein said amount of Chinese lantern oil ranges from 0.001% by weight of the personal care product to 10% by weight of the personal care product.
